(19) United States
(12) Patent Application Publication
(10) Pub. No.: US 2022/0133614 A1
(43) Pub. Date: May 5, 2022

(54) COMPOSITION FOR SKIN WHITENING COMPRISING CARVEDILOL

(71) Applicant: GMEX (GLOBAL MEDICAL XYNAPSE), Seoul (KR)

(72) Inventors: Sung Eun CHANG, Seoul (KR); Young Sup SONG, Seoul (KR); Han Ju YOO, Seoul (KR)

(21) Appl. No.: 17/507,413

(22) Filed: Oct. 21, 2021

(30) Foreign Application Priority Data
Oct. 30, 2020 (KR) 10-2020-0143603

(51) Int. Cl. A61K 8/49; A61Q 19/02; A61K 9/06; A23L 29/00; A61K 31/403

(52) U.S. Cl. CPC A61K 8/492; A61Q 19/02; A61K 31/403; A23L 29/045; A61K 9/06

(57) ABSTRACT

The present invention relates to a composition for skin whitening, and more particularly, to a topical preparation, cosmetic or health functional food composition for skin whitening, which includes carvedilol as an active ingredient. The composition of the present invention, which includes carvedilol as an active ingredient, exhibits a whitening effect by suppressing the expression of a melanin synthesis gene by reducing MITF through inhibition of CREB phosphorylation in normal human melanocytes (NHMs), and gradually weakens cell tyrosinase activity over time, lowering the risk of a side effect of pigmentation. Therefore, the composition is expected to be effectively used in development of materials for cosmetics and health functional food and related industries in safe and effective ways.

### CROSS-REFERENCE TO RELATED APPLICATION

This application claims priority to and the benefit of Korean Patent Application No. 10-2020-0143603, filed on Oct. 30, 2020, the disclosure of which is incorporated herein by reference in its entirety.

### BACKGROUND

### 1. Field of the Invention

The present invention relates to a composition for skin whitening, and more particularly, to a pharmaceutical composition, cosmetic composition, dermal preparation composition, health functional food or quasi-drug for skin whitening, which includes carvedilol as an active ingredient.

### 2. Discussion of Related Art

Pigmented skin diseases are on the rise, and a variety of treatment methods including systemic and topical preparations and laser therapies are being developed. However, these methods have problems of a side effect, such as post-inflammatory hyperpigmentation (PIH), being relatively common and high treatment costs.

Meanwhile, catecholamines consisting of epinephrine and norepinephrine are signaling molecules that act as a neurotransmitter and an endocrine hormone, and the biosynthesis and degradation of a catecholamine in the skin occur in human keratinocytes, but the synthesis of a catecholamine in melanocytes is not well known. Catecholamines act via a G protein-conjugated receptor (GPCR), and binding of a catecholamine with GPCR induces the activation of intracellular adenylate cyclase that synthesizes 3′, 5′-cyclic adenosine monophosphate (cAMP) from ATP. It is known that cAMP binds to an R-subunit of protein kinase A (PKA) to exhibit activity, causing phosphorylation of the cAMP response element binding (CREB) protein, and GPCR is activated by amines including glucagon, parathyroid hormone, secretin and calcitonin and a peptide.

Adrenergic receptor antagonists consist of α-receptor and β-receptor antagonists, in which the α-receptor antagonists are divided into a non-selective α1-selective and α2-selective agents, and the β-receptor antagonists are divided into non-selective, β1-selective and β2-selective antagonists. Unlike first-generation non-selective β-receptor antagonists such as propranolol, timolol and nadolol, carvedilol is known as a third-generation non-selective β-blocker which blocks an α1-blocker to have a vasodilatory action. Similar to other β-blockers, carvedilol is commonly used as an oral drug for regulating high blood pressure and congestive heart disease. In addition, in the field of dermatology, since it is known that an oral preparation of carvedilol has a telangiectasia inhibitory effect, and antioxidant and antiinflammatory effects, it is frequently used to treat erythematotelangiectatic rosacea (Korean Patent No. 1468827).

However, there are few studies confirming the efficacy of carvedilol on skin diseases or whitening, and particularly, nothing is known about signaling processes other than inhibition of an adrenergic receptor are not known, and thus research on this is needed.

### SUMMARY OF THE INVENTION

To improve the above-described problems, the inventors had studied melanin synthesis signaling using carvedilol, confirming that carvedilol inhibits cAMP response element-binding protein (CREB) phosphorylation in human melanocytes, and exhibits a whitening effect by reducing microphthalmia-associated transcription factor (MITF) and inhibiting the expression of a melanin synthesis gene, and thus the present invention was completed.

Therefore, the present invention is directed to providing a composition for skin whitening, which includes carvedilol as an active ingredient.

However, technical problems to be solved in the present invention are not limited to the above-described problems, and other problems which are not described herein will be fully understood by those of ordinary skill in the art from the following description.

To attain the purpose of the present invention, the present invention provides a composition for skin whitening, which includes carvedilol of Formula 1 below as an active ingredient.

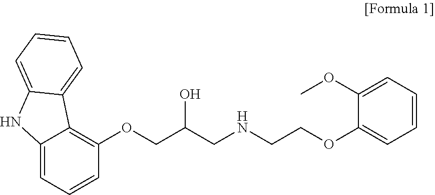 [SMILES: COc1ccccc1OCCNCC(O)COc1cccc2[nH]c3ccccc3c12]

In one embodiment of the present invention, the composition may inhibit MITF subsignals including melanin production, MITF expression and tyrosinase activity.

In another embodiment of the present invention, the composition may reduce MITF expression by the inhibition of CREB phosphorylation.

In still another embodiment of the present invention, the composition may be a topical ointment composition, cosmetic ingredient composition or health functional food composition.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIG. 1 shows a melanin production inhibitory effect according to carvedilol treatment by concentration in normal human melanocytes (NHMs), in which FIG. 1A shows a cell growth rate according to carvedilol treatment by concentration, FIG. 1B shows the melanin production inhibitory activity according to carvedilol treatment by concentration, FIG. 1C shows the inhibition of tyrosinase activity according to carvedilol treatment by concentration, and FIG. 1D shows the expression of a melanin synthesis-related protein according to carvedilol treatment time;

FIG. 2 shows a melanin production inhibitory effect of carvedilol in mouse melanocytes (mel-ab), in which FIG. 2A shows a cell growth rate according to carvedilol treatment by concentration, and FIG. 2B shows a melanin production inhibitory activity according to carvedilol treatment by concentration;

FIG. 3 shows a carvedilol effect in FSK-stimulated NHMs, in which FIG. 3A shows melanin production inhibitory activity over time, FIG. 3B shows MITF mRNA expression according to FSK treatment, and FIG. 3C shows MITF mRNA expression according to carvedilol treatment; and

FIG. 4 shows the change in an ex-vivo skin model according to carvedilol treatment, in which FIG. 4A shows a melanosome level through Fontana-Masson staining, FIG. 4B shows the comparison of a melanin index according to the presence or absence of carvedilol treatment, FIG. 4C shows the change in melanin synthesis-related enzyme, FIG. 4D shows the change in number of melanocytes, and FIG. 4E shows the change in a HMB45(+) melanocyte indicating the activity of the melanocyte.

### DETAILED DESCRIPTION OF EXEMPLARY EMBODIMENTS

As a result of studying melanin synthesis signaling using carvedilol, the inventors confirm that carvedilol inhibits the phosphorylation of the cAMP response element-binding protein (CREB) in normal human melanocytes (NHMs) to reduce microphthalmia-associated transcription factor (MITF) and suppress the expression of a melanin synthesis gene so that a whitening effect is exhibited. Thus, the present invention was completed.

Therefore, the present invention provides a composition for skin whitening, which includes carvedilol as an active ingredient.

In the present invention, the composition for skin whitening may include a pharmaceutical composition, a topical ointment composition, a cosmetic ingredient composition, a dermal preparation composition, a quasi-drug composition or a health functional food composition.

The “carvedilol” used herein is represented by the formula C₂₄H₂₆N₂O₄, and refers to a compound having the structure of Formula 1 below.

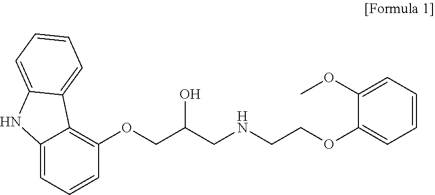 [SMILES: COc1ccccc1OCCNCC(O)COc1cccc2[nH]c3ccccc3c12]

The “active ingredient” used herein refers to a component that is used alone to exhibit desired activity, or used with a non-active carrier to exhibit activity.

The term “whitening” used herein refers to a method of increasing the brightness of the skin with reduced brightness due to an excess of pigments such as melanin or maintaining the brightness of the skin at a certain level, and includes the skin with an increased brightness, formed by the above-described method, and specifically, skin whitening. The “skin whitening” is understood as a result of inhibiting melanin production, and specifically, can be understood as improvement in symptoms, such as melasma and freckles, caused by the increase in melanin, due to the inhibition of melanin production.

The composition of the present invention may exhibit whitening activity by inhibiting melanin production or tyrosinase activity, and specifically, reducing MITF expression through a CREB phosphorylation inhibitory mechanism.

In the present invention, it was confirmed through specific examples that the composition containing carvedilol inhibits melanin production and reduces the expression of a melanin synthesis-related protein, and thus the present invention was completed.

In one embodiment of the present invention, as a result of treating NHMs with carvedilol by concentration, it was confirmed that melanin production was inhibited, tyrosinase activity was inhibited, and the expression of a melanin synthesis-related protein was reduced (see Example 1).

In addition, in another embodiment of the present invention, as a result of comparing the expression of phospho-ERK and phospho-CREB to confirm that the whitening effect of carvedilol is associated with cAMP/PKA/CREB signaling, there was no change in phospho-ERK, but phospho-CREB was reduced over time, demonstrating that whitening activity is exhibited by CREB inhibition by carvedilol (see Example 3).

Accordingly, it was confirmed that the composition of the present invention has a whitening effect by reducing MITF through CREB inhibition, inhibiting the activity of melanocytes and tyrosinase activity, and reducing the expression of a melanin synthesis-related protein, and therefore, the composition containing carvedilol as an active ingredient according to the present invention may be effectively used as a pharmaceutical composition, a topical ointment composition, a cosmetic ingredient composition, a dermal preparation composition, a quasi-drug composition or a health functional food composition.

When the composition according to the present invention is used in the form of a cosmetic composition, in addition to carvedilol or a fraction thereof as an active ingredient, it may further contain a compound or natural extract, which is known to have a skin whitening effect so as to increase or reinforce a skin whitening effect. Here, the compound or natural extract known to have a whitening effect may be mercaptosuccinic acid, mercaptodextran, teprenone, dihydroxy-isoquinoline, indomethacin, 3-hydroxymannule, vitamin K, thiazolidone, kynurenine, lemon extract, cucumber extract, mulberry extract, rosemary extract, acerola cherry extract, gingko extract, or geranium extract, but the present invention is not limited thereto.

Meanwhile, in the cosmetic composition for whitening of the present invention, the active ingredient may be included at any amount (effective amount) depending on a use, formulation or mixing purpose as long as the active ingredient can exhibit skin whitening activity. A conventional effective amount may be included in a range of 0.001 to 99.99 wt % based on the total weight of the composition. Here, the “effective amount” refers to an amount of the active ingredient capable of inducing a whitening effect. The effective amount may be experimentally determined within the ordinary ability of one of ordinary skill in the art.

The cosmetic composition of the present invention may be prepared in various forms, for example, the topical preparation or cosmetic composition of the present invention may be prepared in any formulation conventionally prepared in the art, for example, a solution, a suspension, an emulsion, a paste, a gel, a cream, an ointment, a lotion, a powder, a soap, a cleanser, an oil, a powder foundation, a liquid foundation, a wax foundation, and a spray, but the present invention is not limited thereto. In addition, specifically, the cosmetic composition of the present invention may be prepared in a formulation selected from the group consisting of a skin lotion, a skin softener, a skin toner, an astringent, a lotion, a milk lotion, a moisturizing lotion, a nourishing lotion, a massage cream, a nourishing cream, a moisturizing cream, a hand cream, an essence, a nourishing essence, a pack, a soap, a shampoo, a cleansing foam, a cleansing lotion, a cleansing cream, a body lotion, a body cleanser, an emulsion, a lipstick, a makeup base, a foundation, a pressed powder and a loose powder, but the present invention is not limited thereto.

The topical preparation or cosmetic composition may contain an acceptable carrier in addition to the active ingredient thereof. Here, the “acceptable carrier for a topical preparation or cosmetic preparation” refers to an already known and used compound or composition, which is contained in a cosmetic preparation, or a compound or composition to be developed, which does not have toxicity, instability or irritation beyond a level acceptable to the human body upon contact with the skin. The carrier may be contained at approximately 1 to 99.99 wt %, and preferably, approximately 5 to 99 wt %, with respect to the total weight of the topical preparation or cosmetic composition for whitening of the present invention.

However, since the ratio varies depending on the above-described form of the topical preparation or cosmetic of the present invention, or a specific application site (face or hand) or preferable application amount thereof, the ratio should not be construed as limiting the scope of the present invention in any aspect.

Meanwhile, as the carrier, an alcohol, an oil, a surfactant, a fatty acid, a silicone oil, a wetting agent, a viscosity modifier, an emulsion, a stabilizer, a sunscreen, a coloring agent, or a fragrance may be exemplified. Since compounds/compositions that can be used as an alcohol, an oil, a surfactant, a fatty acid, a silicone oil, a wetting agent, a viscosity modifier, an emulsion, a stabilizer, a sunscreen, a coloring agent, or a fragrance, which can be used as the carrier, are already known in the art, a suitable corresponding material/composition may be selected and used by one of ordinary skill in the art.

The composition according to the present invention may be used in the form of a topical preparation, and the “topical preparation” is a concept encompassing all materials generally used for topical use, non-limiting examples of a formulation for topical use include a plaster, a lotion, a liniment, a liquid and a solution, an aerosol, an extract, an ointment, a fluid extract, an emulsion, a suspension, a capsule, a cream, a soft or hard gelatin capsule, a patch, or a sustained release formulation. The topical preparation according to the present invention may be a parenteral preparation formulated in a solid, semi-solid or liquid form by adding a commercially available inorganic or organic carrier, excipient or diluent. The preparation for parenteral administration may be a form for transdermal administration selected from the group consisting of a drop, an ointment, a lotion, a gel, a cream, a patch, a spray, a suspension and an emulsion, but the present invention is not limited thereto.

When the composition of the present invention is used as a pharmaceutical composition, it may have a function of treating a pigment deposited on the skin, and a function of helping skin whitening by inhibiting the occurrence of a skin pigmentation disease by preventing the excessive deposition of melanin pigment on the skin. The pharmaceutical composition of the present invention can be formulated in the form of a tablet, a pill, a powder, granules, a capsule, a suspension, an oral liquid, an emulsion, a syrup, an aerosol, or a sterile injectable solution according to a conventional method for preventing and treating skin pigmentation. Various specific doses of the pharmaceutical composition according to the present invention may be selected by one of ordinary skill in the art according to factors such as a preparation method, a patient's condition, weight, gender and age, the severity of a disease, a drug form, an administration route and duration, an excretion rate, and reaction sensitivity, and the dosage and frequency do not limit the scope of the present invention in any way.

When the composition according to the present invention is used as a health functional food composition, the health functional food of the present invention may be prepared in various formulations. Unlike general medicines, the health functional food of the present invention using food as a raw material has no side effect that may occur when taking a drug for a long time and is excellent in portability, and therefore, the health functional food of the present invention can be taken as a supplement for improving a skin whitening effect.

The health functional food composition according to the present invention may be prepared as food with high medical and therapeutic effects, which is processed to efficiently exhibit a bioregulatory function, in addition to food for specific use and nutritional supply, and the food may be used interchangeably with functional food, health food and health supplement food in some cases, and may be prepared in various forms such as a tablet, a capsule, a powder, granules, a liquid and a pill.

The health functional food of the present invention may include additional ingredients that are commonly used in food compositions to improve smell, taste, appearance and the like. For example, the health functional food may include vitamin A, C, D, E, B1, B2, B6 or B12, niacin, biotin, folate, or panthotenic acid. In addition, the health functional food may include a mineral such as zinc (Zn), iron (Fe), calcium (Ca), chromium (Cr), magnesium (Mg), manganese (Mn) or copper (Cu). The health functional food may also include an amino acid such as lysine, tryptophan, cysteine or valine. To the health functional food of the present invention, food additives such as a preservative (potassium sorbate, sodium benzoate, salicylic acid, dihydroacetate, or the like), an antibacterial agent (bleaching powder, hypochlorite powder, sodium hypochlorite, or the like), an antioxidant (butylhydroxyanisole (BHA), butylhydroxytoluene (BHT), or the like), a pigment (tar pigment or the like), a coloring agent (sodium nitrite, sodium acetate, or the like), a bleaching agent (sodium sulfite or the like), a seasoning (monosodium glutamate (MSG) or the like), a sweetener (dulcin, cyclamic acid, saccharin, sodium, or the like), a flavor (vanillin, lactones, or the like), a swelling agent (alum, D-potassium hydrogen tartrate, or the like), a reinforcing agent, an emulsifier, a thickener (thickening agent), a film-forming agent, a gum base, a foam inhibitor, a solvate or an improving agent, may be added. The additives may be selected according to the type of food, and may be used at suitable amounts.

When the health functional food of the present invention is used as a food additive, it may be added alone or used in combination with another food or food ingredient, and may be suitably used according to a conventional method.

In the health functional food of the present invention, the content of carvedilol may vary according to the condition of a subject to be administered, the type of specific disease, and the degree of progression, or the like. When needed, the carvedilol may be included at the total content of the food.

Hereinafter, the present invention will be described in further detail through examples. However, the following examples are merely provided to illustrate the present invention, and the scope of the present invention is not limited to these examples.

### EXAMPLES

### Example 1. Confirmation of Whitening Effect According to Carvedilol Treatment in Melanocytes

1-1. Confirmation of Whitening Effect According to Carvedilol Treatment in Normal Human Melanocytes (NHMs)

NHMs were treated with carvedilol at each concentration of 2 μM, 4 μM, 6 μM, 8 μM and 10 μM, cultured for 5 days, and then a cell growth rate, melanogenesis activity, tyrosinase activity, and the expression of a melanin synthesis-related protein were confirmed.

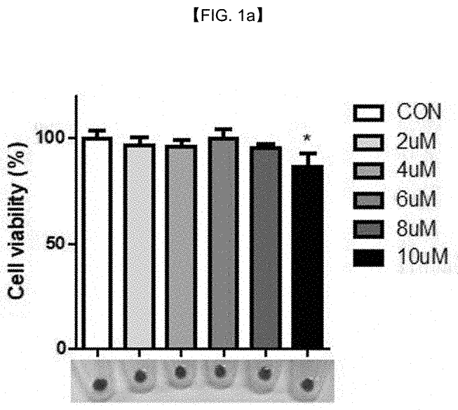

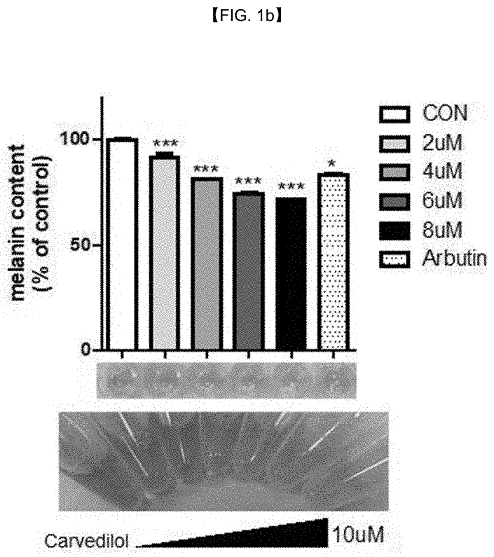

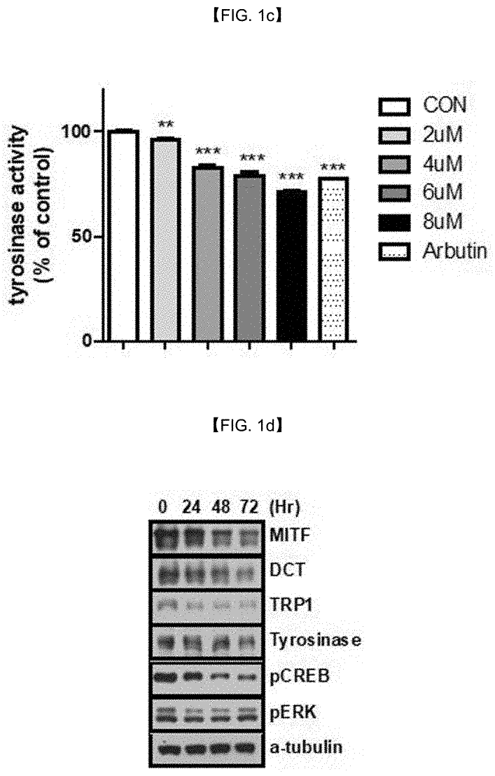

As a result, as shown in FIG. 1A, in the case of the cell growth rate, it was confirmed that there was no effect up to 8 μM carvedilol, and in the case of 10 μM carvedilol, the cell growth rate was slightly reduced. In addition, as shown in FIGS. 1B and 1C, melanin production and tyrosinase activity were inhibited by carvedilol treatment. In addition, as shown in FIG. 1D, it was confirmed that the expression of a melanin synthesis-related protein tends to decrease over time.

1-2. Confirmation of Melanin Production Inhibitory Effect According to Carvedilol Treatment in Mouse Melanocytes (Mel-Ab)

In addition to the result of Example 1 showing whitening activity in NHMs, the melanin inhibitory effect was confirmed by treating mouse melanocytes (mel-ab) with carvedilol.

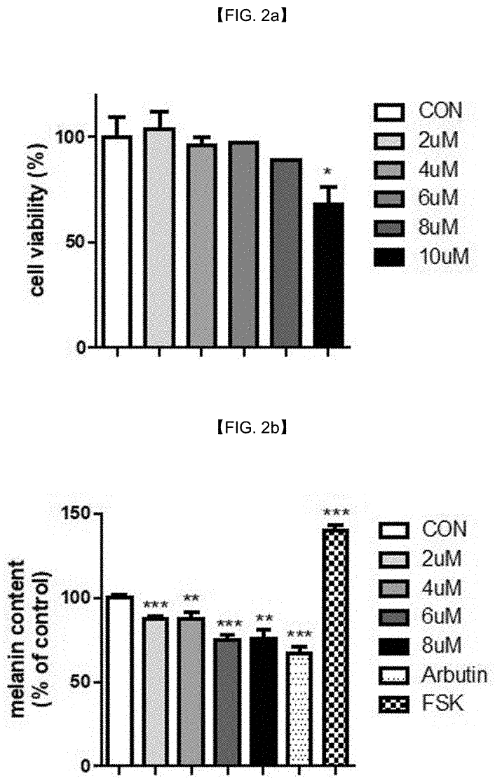

As a result, as shown in FIGS. 2A and 2B, even in mouse melanocytes (mel-ab), it was confirmed that there was no effect on the cell growth rate up to 8 μM carvedilol, but there was an effect of inhibiting melanin production.

### Example 2. Confirmation of Whitening Effect According to Carvedilol Treatment in FSK-Simulated NHMs

After NHMs were stimulated with FSK and treated with carvedilol, the expression levels of phospho-CREB and phospho-ERK were measured, and an intracellular signaling pathway which regulates MITF transcription was investigated.

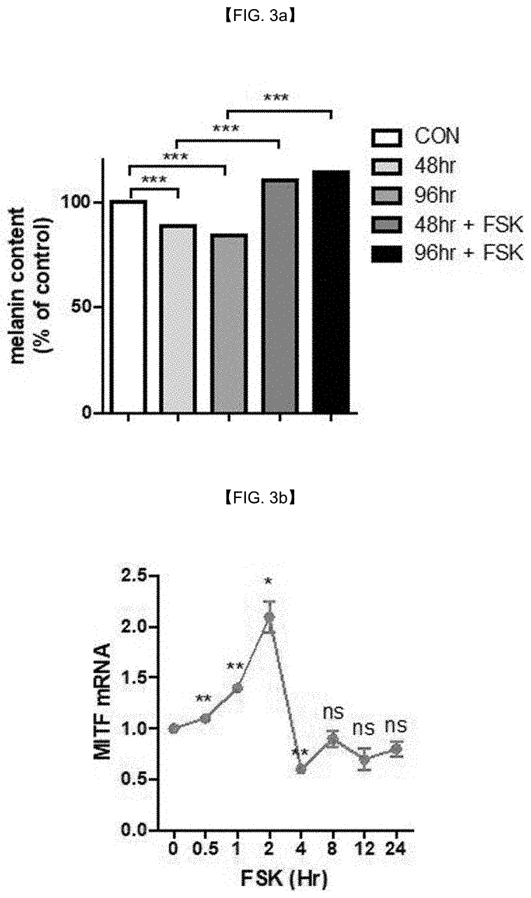

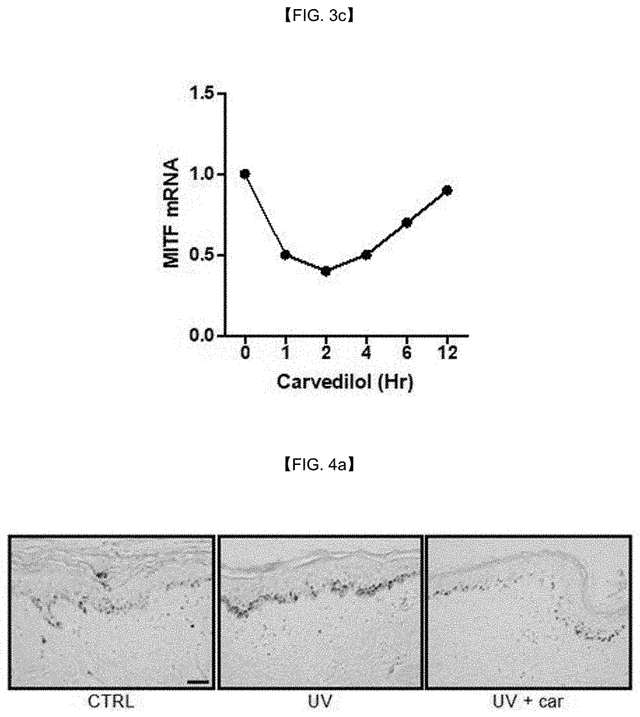

As a result, as shown in FIG. 1D, it was confirmed that there was no change in phospho-ERK, but the expression of phospho-CREB was reduced over time. Therefore, it was assumed that carvedilol inhibits the cAMP/PKA/CREB signaling pathway, thereby inhibiting melanin production, and as a result of re-confirming a melanin production inhibitory effect of carvedilol after FSK increasing cAMP was treated, as shown in FIG. 3A, it was confirmed that carvedilol inhibits melanin production.

In addition, as shown in FIG. 3B, it was confirmed that, after the mRNA of MITF having its own structural response curve for cell survival and biological functions was treated with 10 μM FSK for 2 hours, MITF transcription maximally increased in NHMs. However, as shown in FIG. 3C, it was confirmed that the mRNA level of MITF maximally decreased in NHMs two hours after carvedilol treatment.

### Example 3. Confirmation of Change in Ex-Vivo Skin Model According to Carvedilol Treatment

To examine adaptation in actual clinical practice, an ex-vivo model of human skin tissue was stimulated by UV irradiation to induce melanin synthesis, and treated with carvedilol and cultured, and then a fragment was formed and subjected to Fontana-Masson staining.

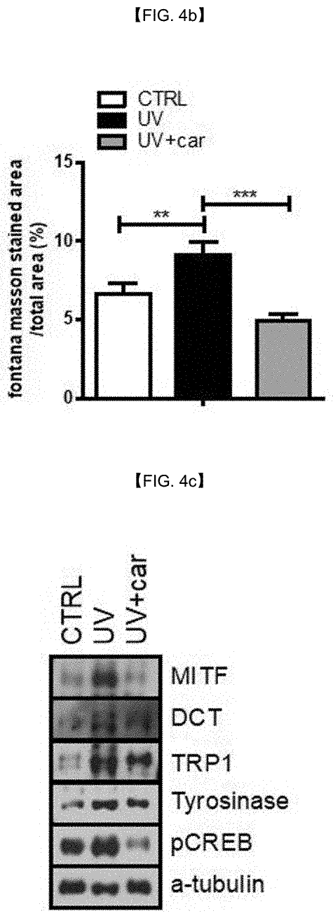

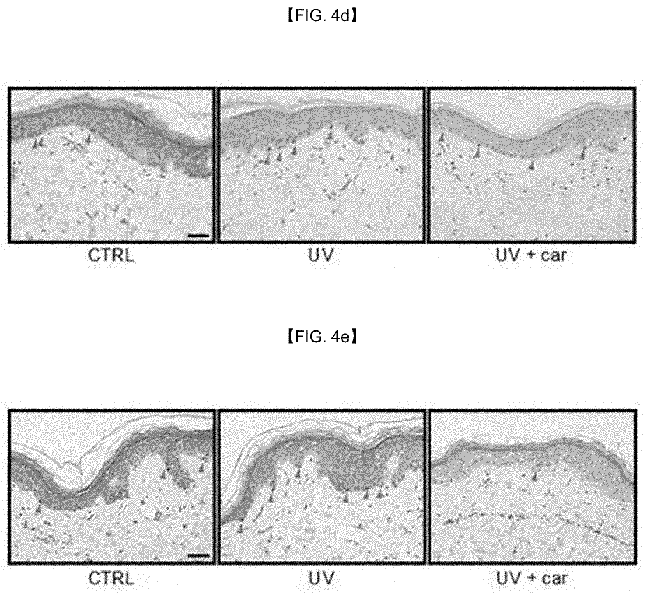

As a result, as shown in FIG. 4A, a melanosome reduction was confirmed, and as a result of calculating a proportion of a Fontana-Masson-stained region, as shown in FIG. 4B, a decrease in melanin index when carvedilol is treated was confirmed, and as shown in FIG. 4C, it was also confirmed that melanin synthesis-related enzymes such as tyrosinase, TRP1 and DCT were increased by UVR, and reduced by carvedilol treatment.

In addition, as a result of analyzing the tissue immunostaining result, as shown in FIG. 4D, it was confirmed that carvedilol does not affect the number of Melan-A (+) cells, that is, melanocytes, compared to UVR alone. In addition, as shown in FIG. 4E, HMB45(+) melanocytes indicating melanocyte activity were increased by UVR treatment, and reduced by carvedilol treatment.

From the above result, it was confirmed that carvedilol significantly reduces a melanin index and the expression of a melanin production-related protein, exhibiting a melanin production inhibitory effect on human skin irritated by UVR.

A composition containing carvedilol as an active ingredient according to the present invention can exhibit a whitening effect of suppressing the expression of a melanin synthesis gene by reducing microphthalmia-associated transcription factor (MITF) by inhibiting the phosphorylation of CREB in normal human melanocytes (NHMs), and gradually weaken cell tyrosinase activity over time, lowering the risk of side effects of pigmentation, and therefore, it is expected that the composition of the present invention can be effectively used in development of materials for cosmetics and health functional food and related industries in safe and effective ways.

It should be understood by those of ordinary skill in the art that the above descriptions of the present invention are exemplary, and the example embodiments disclosed herein can be easily modified into other specific forms without changing the technical spirit or essential features of the present invention. Therefore, it should be interpreted that the example embodiments described above are exemplary in all aspects, and are not limitative.

1. A composition for skin whitening, comprising carvedilol of Formula 1 below as an active ingredient:

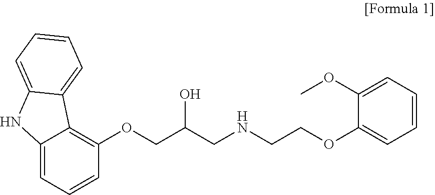 [SMILES: COc1ccccc1OCCNCC(O)COc1cccc2[nH]c3ccccc3c12]

2. The composition of claim 1, wherein the composition inhibits MITF subsignals including melanin production, MITF expression and tyrosinase activity.

3. The composition of claim 1, wherein the composition reduces the expression of microphthalmia-associated transcription factor (MITF) through the inhibition of cAMP response element-binding protein (CREB) phosphorylation.

4. The composition of claim 1, wherein the composition is a cosmetic composition.

5. The composition of claim 1, wherein the composition is a health functional food composition.

6. The composition of claim 1, wherein the composition is a topical ointment composition.
